(19) United States
(12) Patent Application Publication
(10) Pub. No.: US 2026/0034331 A1
(43) Pub. Date: Feb. 5, 2026

(54) SYSTEMS AND METHODS FOR OPTIMIZING NEURAL STIMULATION BASED ON MEASURED NEUROLOGICAL SIGNALS

(71) Applicant: OSCILLOSCAPE, LLC, East Hartford, CT (US)

(72) Inventors: Edward W. Large, East Hartford, CT (US); Ji Chul Kim, East Hartford, CT (US)

(73) Assignee: OSCILLOSCAPE, LLC, East Hartford, CT (US)

(21) Appl. No.: 19/242,478

(22) Filed: Jun. 18, 2025

Related U.S. Application Data
(63) Continuation of application No. PCT/US2023/083623, filed on Dec. 12, 2023.
(60) Provisional application No. 63/433,547, filed on Dec. 19, 2022.

(51) Int. Cl. A61M 21/00; G06F 3/01

(52) U.S. Cl. CPC A61M 21/00; G06F 3/015; A61M 2021/0027; A61M 2021/005; A61M 2210/0693

(57) ABSTRACT

A system comprising an output device, a brain oscillation monitor, and one or more processors. The output device is configured to output an audio signal for audio stimulation of the patient, and a visual pattern for video stimulation of the patient. The brain oscillation monitor is configured to generate feedback indicative of a response to the audio stimulation and the visual stimulation. The one or more processors are configured to determine a target frequency for the audio stimulation and the visual stimulation. The one or more processors are further configured to cause constructive interference of the audio stimulation and the visual stimulation output by the output device at the target frequency, by modifying at least one of a phase of the visual pattern or an amplitude of onsets for the visual pattern, relative to the audio signal, according to the feedback from the brain oscillation monitor.

### CROSS-REFERENCE TO RELATED APPLICATIONS

This application claims priority to U.S. Provisional Patent Application No. 63/433,547, filed Dec. 19, 2022, the content of which is incorporated by reference in its entirety.

### FIELD OF DISCLOSURE

The present disclosure is generally related to neural stimulation including, but not limited to, systems and methods for optimizing neural stimulation based on measured neural stimulation.

### BACKGROUND

Neural oscillation occurs in humans and animals and includes rhythmic or repetitive neural activity in the central nervous system. Neural tissue can generate oscillatory activity by mechanisms within individual neurons or by interactions between neurons. Oscillations can appear as either periodic fluctuations in membrane potential or as rhythmic patterns of action potentials, which can produce oscillatory activation of post-synaptic neurons. Synchronized activity of a group of neurons can give rise to macroscopic oscillations, which can be observed by sensing electrical or magnetic fields in the brain using techniques such as electroencephalography (EEG), intracranial EEG (iEEG), also known as electrocorticography (ECoG), and magnetoencephalography (MEG).

### SUMMARY

According to the systems and methods described herein, neural stimulation can be provided via rhythmic light stimulation that is presented simultaneously with auditory stimulation through music. The combination of music and light stimuli can elicit neural oscillation effects or stimulation. The combined stimuli can adjust, control or otherwise affect the frequency of the neural oscillations to provide beneficial effects to one or more cognitive states, cognitive functions, the immune system or inflammation, while mitigating or preventing adverse consequences on a cognitive state or cognitive function. For example, systems and methods of the present technology can treat, prevent, protect against or otherwise affect Alzheimer's Disease or other cognitive diseases, such as Parkinson's disease, dementia, and so forth.

In various instances, where a patient is undergoing treatment or is otherwise undergoing both audio and visual stimulation as described herein, often times that stimulation is at a targeted or particular frequency or frequency band (e.g., in the delta, theta, and/or gamma band) to stimulate a particular portion of the patient's brain. However, even if the audio stimulation is at the same frequency as the visual stimulation, the resultant brain signals from the audio stimulation and visual stimulation may not arrive at the target portion of the patient's brain at the same time. In this regard, while the audio and visual stimulation may be at substantially the same frequency, the audio and visual stimulations may be out of phase. To the extent that the audio and visual stimulations are out of phase, the resultant brain signals may destructively interfere with each other, thereby reducing the efficacy of stimulation and treatment. On the other hand, to the extent that the audio and visual stimulations are in phase with one another, the resultant brain signals may constructively interfere with each other, thereby amplifying the stimulation in the target portion of the patient's brain.

In various embodiments, and as described in greater detail below, the systems and methods described herein may be configured to modify, update, change, or otherwise shift a phase of the audio and/or visual stimulation, to phase align the audio stimulation and the visual stimulation. By shifting a phase of the audio and/or visual stimulation such that the resultant brain signals constructively interfere with one another, the systems and methods described herein may amplify the stimulation in the target portion of the patient's brain, thereby increasing the efficacy of stimulation and treatment.

In various aspects, this disclosure is directed to systems and methods for optimizing neural stimulation based on measured neurological signals. An output device may be configured to output an audio signal for audio stimulation of the patient, and a visual pattern for video stimulation of the patient. A brain oscillation monitor may be configured to generate feedback indicative of a response to the audio stimulation and the visual stimulation. One or more processors may be configured to determine a target frequency for the audio stimulation and the visual stimulation. The one or more processors may be configured to cause constructive interference of the audio stimulation and the visual stimulation output by the output device at the target frequency, by modifying at least one of a phase of the visual pattern or an amplitude of onsets for the visual pattern, relative to the audio signal, according to the feedback from the brain oscillation monitor.

In some embodiments, the one or more processors are configured to determine, using the feedback from the brain oscillation monitor, an amplitude in the response at the target frequency. In some embodiments, the one or more processors are configured to modify the at least one of the phase or the amplitude, responsive to the amplitude in the response at the target frequency satisfying a threshold criteria. In some embodiments, the one or more processors are configured to modify each of the phase of the visual pattern and the amplitude of the onsets for the visual pattern, relative to the audio signal.

### BRIEF DESCRIPTION OF THE DRAWINGS

The accompanying drawings are not intended to be drawn to scale. Like reference numbers and designations in the various drawings indicate like elements. For purposes of clarity, not every component can be labeled in every drawing.

FIG. 1 is a diagram illustrating the frequencies selected by an oscillation selection module (OSM) as they relate to a specific underlying musical stimulus, and the range of frequencies present in each frequency band, according to an example implementation of the present disclosure.

FIG. 2 is a diagram illustrating, on the left hand side, magnetoencephalography (MEG) recordings of human auditory cortex recorded while subjects listened to rhythmic auditory stimuli at two different tempos, and on the right hand side, highlights of some of the brain areas that exhibited this response.

FIG. 3 is a block diagram of a system for providing neurological stimulation, according to an example implementation of the present disclosure.

FIG. 4 is a diagram showing operation of the system of FIG. 3 with resultant brain stimuli, according to an example implementation of the present disclosure.

FIG. 5-FIG. 6 are diagrams showing example stimulus provided the system of FIG. 3, using different songs, where Panel A compares the auditory rhythmic frequencies (i.e., the onset spectrum) of the music with the frequency of an auditory 40 Hz pulse train, and Panel B compares the visual frequencies stimulated by the system with the frequency of a visual 40 Hz pulse train, according to an example implementation of the present disclosure.

FIG. 7 is a diagram of an output device for delivering visual stimulation, according to an example implementation of the present disclosure.

FIG. 8 is a block diagram of an example computer system, according to an example implementation of the present disclosure.

### DETAILED DESCRIPTION

Before turning to the figures, which illustrate certain embodiments in detail, it should be understood that the present disclosure is not limited to the details or methodology set forth in the description or illustrated in the figures. It should also be understood that the terminology used herein is for the purpose of description only and should not be regarded as limiting.

Neural oscillations can be characterized by their frequency, amplitude, and phase. These signal properties can be observed from neural recordings using time-frequency analyses. For example, an EEG can measure oscillatory activity among a group of neurons, and the measured oscillatory activity can be categorized into frequency bands as follows: delta activity corresponds to a frequency band from 0.5-4 Hz; theta activity corresponds to a frequency band from 4-8 Hz; alpha activity corresponds to a frequency band from 8-13 Hz; beta activity corresponds to a frequency band from 13-30 Hz; and gamma activity corresponds to a frequency band of 30 Hz and above.

Neural oscillations of different frequency bands can be associated with cognitive states or cognitive functions such as perception, action, attention, reward, learning, and memory. Based on the cognitive state or cognitive function, the neural oscillations in one or more frequency bands may be involved. Further, neural oscillations in one or more frequency bands can have beneficial effects or adverse consequences on one or more cognitive states or functions.

Neural entrainment occurs when an external stimulation of a particular frequency or combination of frequencies is perceived by the brain and triggers neural activity in the brain that results in neurons oscillating at frequencies related to the particular frequencies of the external stimulation. Thus, neural entrainment can refer to synchronizing neural oscillations in the brain using external stimulation such that the neural oscillations occur at the frequencies corresponding to the particular frequencies of the external stimulation. Neural entrainment can also refer to synchronizing neural oscillations in the brain using external stimulation such that the neural oscillations occur at frequencies that correspond to harmonics, subharmonics, integer ratios, and combinations of the particular frequencies of the external stimulation. The specific neural oscillatory frequencies that can be observed in response to a set of external stimulation frequencies are predicted by models of neural oscillation and neural entrainment.

Cognitive functions such as learning and memory involve coordinated activity across distributed subcortical and cortical brain regions, including hippocampus, cortical and subcortical association areas, sensory regions, and prefrontal cortex. Across different brain regions, behaviorally relevant information is encoded, maintained, and retrieved through transient increases in the power of and synchronization between neural oscillations that reflect multiple frequencies of activity.

In particular, oscillatory neural activity in the theta and gamma frequency bands are associated with encoding, maintenance, and retrieval processes during short-term, working, and long-term memory. Induced gamma activity has been implicated in working memory, with increases in scalp-recorded and intracranial gamma-band activity occurring during working-memory maintenance. Increases in the power of gamma activity dynamically track the number of items maintained in working memory. Using electrocorticography (ECoG), one study found enhancements in gamma power tracked working-memory load in the hippocampus and medial temporal lobe, as participants maintained sequences of letters or faces in working memory. Finally, other evidence indicates that hippocampal gamma activity aids episodic memory, with distinct sub-gamma frequency bands corresponding to encoding and retrieval stages.

Theta oscillations (4-8 Hz) have been linked to working and episodic memory processes. Intracranial EEG (iEEG) recordings demonstrate that, during working memory, theta oscillations gate on and off (i.e., increase and sustain in amplitude, before rapidly decreasing in amplitude) over the encoding, maintenance, and retrieval stages. Other work has observed increases in scalp-recorded theta activity during working-memory maintenance. Some studies have concluded that scalp-recorded theta activity, emerging from frontal-midline electrodes, was the most robust neural correlation of verbal working-memory maintenance. Moreover, frontal-midline theta activity tracks working-memory load, increasing and sustaining in power as a function of the number of items maintained in working memory.

Some studies have found that gamma-frequency, auditory-visual stimulation can ameliorate dementia or Alzheimer's Disease (AD)-related biomarkers and pathophysiologies, and, if administered during an early stage of disease progression, can provide neuroprotection.

Music entrains and drives neural activity in multiple frequency ranges, and musical stimulation itself can entrain and drive oscillatory neural activity that is involved in learning, memory, and cognition. In various embodiments of the present solution, the systems and methods described herein may detect, determine, identify, or otherwise leverage on the brain's natural delta, theta, and gamma frequency responses to music, by providing music as the sole auditory stimulus in a system and method for treating, preventing, protecting against or otherwise affecting Alzheimer's Disease, dementia, and/or other neurological or cognitive conditions. In some embodiments, the audio stimulus is coupled with visual stimulation in the delta, theta, and/or gamma frequency bands, which is choreographed to synchronize with the delta, theta and/or gamma frequency bands of the brain's response to the audio stimulus for enhanced therapeutic effect. In some embodiments, additional frequencies and frequency bands can be targeted for stimulation, to treat, prevent, and/or protect against Alzheimer's Disease, dementia, and/or other neurological or cognitive conditions or ailments, such as Parkinson's Disease.

Musical rhythms are organized into well-structured frequency combinations. For example, musical rhythms entrain neural activity in the delta and theta frequency ranges, by directly stimulating the brain at these frequencies. The frequency of the basic beat may correspond to neural activity in the delta frequency band. Subdivisions of the beat typically correspond to neural activity in the theta frequency band. Additionally, musical rhythms can drive activity at delta and theta frequencies that are not explicitly present in the rhythms, because musical rhythms contain structured frequency combinations. Frequencies observed in brain activity can include harmonics, subharmonics, integer ratios, and combinations of frequencies present in the musical rhythms, and are predicted by simulations of neural oscillation and neural entrainment.

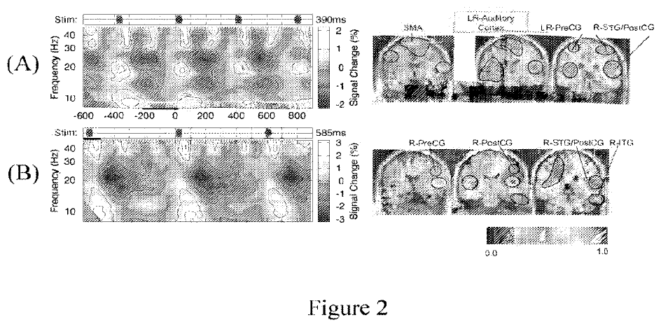

Musical rhythms can drive gamma neural activity in the brain in a way that is different than the entrainment of delta and theta activity. The amplitude of endogenous gamma neural oscillations is modulated such that amplitude peaks synchronize with musical events (see FIG. 2). Amplitude modulation of gamma neural activity reflects phase-amplitude coupling to lower frequency (e.g., delta and theta) neural activity.

Phase-amplitude coupling (PAC) may be or include a statistical dependency between the amplitude of oscillations in one frequency band and the phase of oscillations in another frequency band. For example, in theta-gamma phase-amplitude coupling, peaks in gamma amplitude correspond to a specific phase of entrained theta activity. Thus, gamma activity is driven by entrained theta and delta activity.

The systems and methods described herein may provide feedback-based audio and/or visual stimulation by activating the brain's natural delta, theta, and gamma responses to music in a way that does not interfere with musical enjoyment. Because enjoyment is critical for patient tolerability and completion of protocols, the systems and methods described herein may incentivize patient compliance with the treatment by avoiding the abrasive and unpleasant sounds of added audio waves in the gamma frequency band.

In some embodiments, the systems and methods described herein may incorporate, produce, or otherwise provide visual stimulation in the delta, theta, and/or gamma frequency bands, so as to enhance the frequencies that are important in musical enjoyment. Such solutions may enhance the efficacy of stimulation because visual stimulation in the gamma band is less aversive than auditory stimulation in the gamma band. In some embodiments, gamma stimulation can be combined with delta and theta stimulation, to create visual stimulation that mimics the brain's natural response to musical rhythms.

In the systems and methods described herein, gamma stimulation can be amplitude-modulated through phase-amplitude coupling to theta and/or delta frequency oscillations to mimic auditory processing, increasing the efficacy and extent of neural stimulation. Furthermore, the specific stimulus frequencies are determined by the musical stimuli, and so stimulus frequencies provided by the present solution change within a stimulus session, decreasing the potential for neural adaptation, and thus increasing stimulus efficacy. In some embodiments, the systems and methods described herein may combine music listening with delta, theta, and/or gamma frequency visual stimulation to create engaging, and effective audiovisual stimuli for patients. In some embodiments, additional frequency bands may be employed, both via audio or visual stimuli.

In some embodiments, the systems and methods described herein may output an improved set of stimuli which amplify the brain's natural delta, theta, and gamma responses to music in a way that does not create neural interference between the brain's natural oscillatory responses to music and added oscillatory auditory stimulation within the same frequency bands. Specifically, in some embodiments, the systems and methods described herein may use a simulation of neural entrainment to determine the frequencies of the brain's natural delta, theta, and gamma responses to music. The system may then reinforce and amplify the natural responses to music by delivering the same delta, theta, and/or gamma frequencies in visual stimulation. The simulation can include delta-theta-gamma phase-amplitude coupling to faithfully mimic the brain's auditory response, and amplify the effect. Thus, the visual stimulation may not interfere with, or cancel, the brain's natural oscillatory responses to music. Rather, the visual stimulation may amplify the brain's natural oscillatory responses to the music.

The systems and methods described herein are directed to outputting stimuli which elicit neural stimulation via rhythmic light stimulation that is presented simultaneously with musical stimulation. The combination of music and rhythmic light pulses can elicit brainwave effects or stimulation. The combined stimuli can adjust, control, or otherwise affect the frequency of the neural oscillations to provide beneficial effects to one or more cognitive states, cognitive functions, the immune system or inflammation (or other conditions), while mitigating or preventing adverse consequences on a cognitive state or cognitive function, and maximizing enjoyment, treatment tolerability, and completion of treatment protocol. For example, systems and methods of the present technology can treat, prevent, protect against, or otherwise affect Alzheimer's Disease (or other cognitive diseases or ailments).

The frequencies of neural oscillations observed in patients can be affected by or correspond to the frequencies of the musical rhythm and the rhythmic light pulses. Thus, systems and methods of the present solution can elicit neural entrainment by outputting multi-modal stimuli such as musical rhythms and light pulses emitted at frequencies determined by analysis of the musical rhythm. This combined, multi-modal stimulus can synchronize electrical activity among groups of neurons based on the frequency or frequencies that are entrained and driven by musical rhythm. Neural entrainment can be observed based on the aggregate frequency of oscillations produced by the synchronous electrical activity in ensembles of neurons throughout the brain.

In some embodiments, additional outputs from the system may also include one or more stimulation units for generating tactile, vibratory, thermal and/or electrical transcutaneous stimuli. Such stimulation units may include a mobile device, smart watch, gloves, or other devices that can vibrate. In some embodiments, the output device may include stimulation units for generating electromagnetic fields or electrical currents, such as an array of electromagnets or electrodes, to deliver transcranial stimulation.

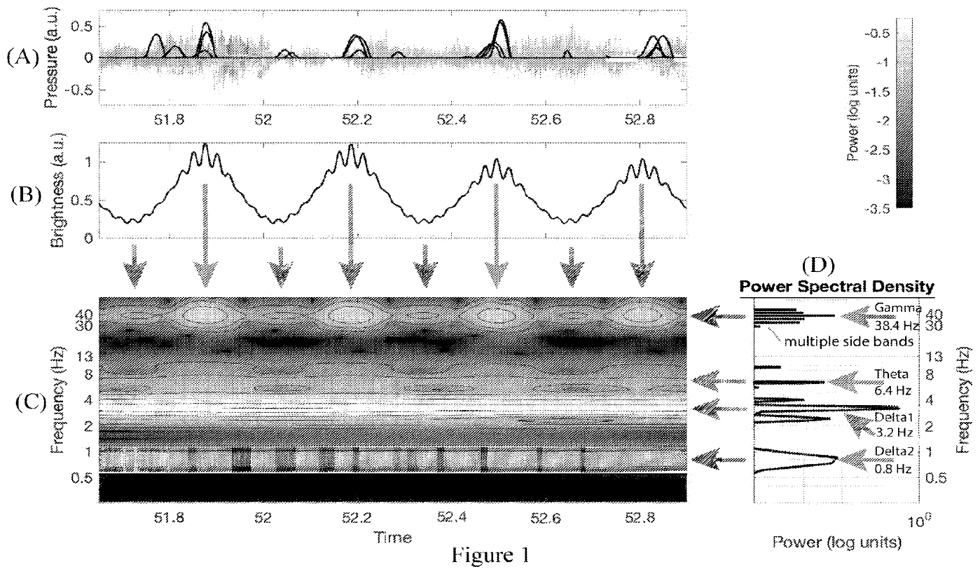

Referring to FIG. 1, depicted is a diagram illustrating the frequencies selected by an oscillation selection module (OSM) as they relate to a specific underlying musical stimulus, and the range of frequencies present in each frequency band, according to an example implementation of the present disclosure. As shown in FIG. 1, the diagram may include a breakdown of four frequencies that can be selected by the systems and methods described herein they relate to the underlying music, and the range of frequencies present. In some embodiments, the systems and methods described herein may select one or more harmonically related frequencies in the delta, theta, and lower gamma (30-50 Hz) frequency ranges. In some embodiments, the gamma amplitude is modulated by the theta frequency, simulating theta-gamma phase-amplitude coupling. Also in some embodiments, the theta amplitude is modulated by one or more delta frequencies, simulating the delta-theta phase amplitude coupling. Collectively, the foregoing, thereby simulates the delta-theta-gamma oscillatory hierarchy in the auditory cortex.

With continued reference to FIG. 1, there is illustrated an exemplary protocol for visual stimulation frequencies produced by the system in the gamma, theta, and delta frequency bands according to an aspect of the present disclosure. Panel A shows the time-domain waveform of the music stimulus over a 4-beat time interval, and the onsets computed during preprocessing. Panel B shows the delta-theta-gamma coupled changes in brightness provided by the systems and methods described herein, while Panel C shows the same changes in each frequency band.

FIG. 2 shows an MEG recording of a human auditory cortex recorded while the subject listened to two rhythms with different tempos. Panel A of FIG. 2 is a time-frequency map of signal power changes related to rhythmic stimulus presented every 390 ms (2.6 Hz), which shows a periodic pattern of signal increases and decreases in the gamma frequency band. Panel B shows the same measurement with respect to a rhythmic stimulus presented every 585 ms (1.7 Hz). In the auditory cortex, gamma is amplitude modulated by delta and theta, and this pattern is simulated by the systems and methods described herein.

Panel D of FIG. 1 illustrates the stimulus produced by the systems and methods described herein in the frequency domain. Collectively, these figures illustrate that gamma oscillations are effectively stimulated by the output provided by the device in a range of frequencies around the main frequency. These additional frequencies are called sidebands, and they are caused by the device and method's amplitude modulation from theta and delta frequencies. Moreover, each song played by the systems and methods described herein leads to a different choice of frequencies within the delta, theta, and gamma ranges. Thus, over the course of several songs played via the systems and methods described herein, the output stimulates many gamma frequencies.

The device thus simulates an amplitude modulation of the stimulus provided in the gamma frequency band by the phase of stimulation provided in the delta and theta frequency bands, which mimics the brain's natural gamma-delta-theta phase-amplitude coupling response and thereby enhances both tolerance and efficacy of the treatment. As noted above, Panel D of FIG. 1 shows that gamma oscillations are effectively stimulated in a range of frequencies (sidebands) around the main frequency. These sidebands are caused by the amplitude modulation from theta and delta frequencies provided by the systems and methods described herein.

Moreover, each musical composition played by the system may lead to a different choice of frequencies within the delta, theta, and gamma ranges. Thus, over the course of one session, different gamma frequencies are stimulated. By contrast, some solutions may only stimulate one frequency, and a common outcome is neural adaptation, leading to a reduced neural response. In some embodiments of the present system, changing frequencies may avoid neural adaptation and promote robust neural responses.

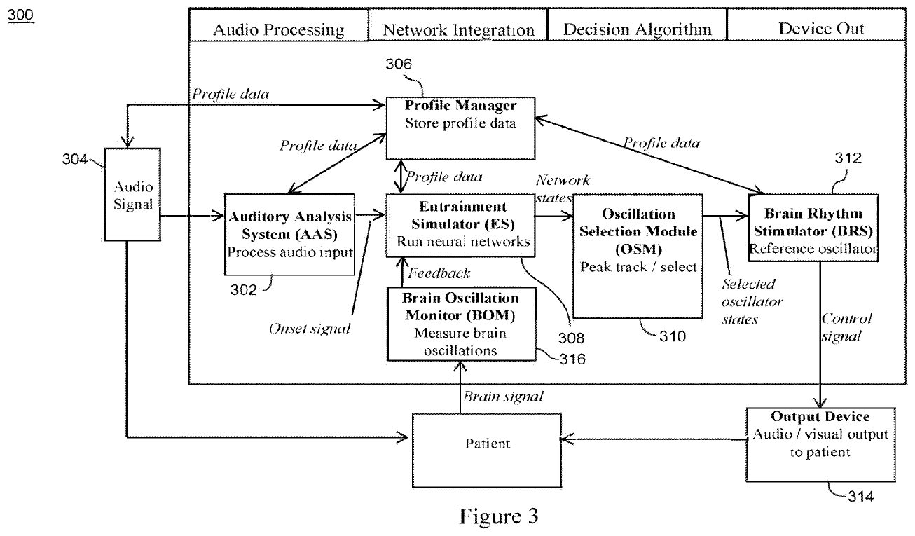

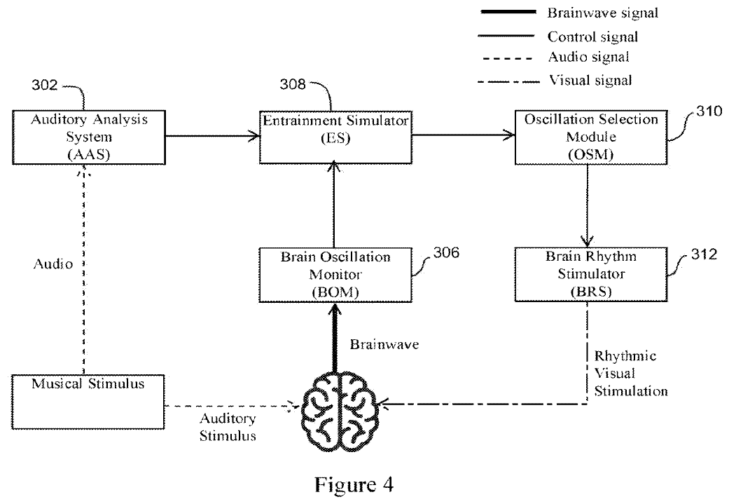

Referring now to FIG. 3 and FIG. 4, depicted is a block diagram of a system 300 for providing neurological stimulation, and a diagram showing operation of the system 300 with resultant brain stimuli, according to example implementations of the present disclosure. The system 300 may include an Auditory Analysis System (AAS) 302 configured to receive auditory input, filter the acoustic signal, detect the onset of acoustic events (e.g., notes or drum hits) and adjust the gain of the resulting signal. In some embodiments, the AAS 302 may include a filtering module, an onset detection module, and an optional gain control module to filter a signal, detect the onset of acoustic events, and adjust a gain of the resulting signal, respectively.

In some embodiments, the AAS 302 may be configured to pre-process an auditory stimulus, auditory input, or audio signal 304, to provide multi-channel rhythmic inputs (e.g., note onsets). In some embodiments, the auditory input or audio signal 304 is provided by the system, such as by or via a built-in audio playback system that has access to a library of songs and/or other musical compositions. In some embodiments, the system 300 may further comprise a graphical display and input/output accessible to the user (e.g. patient or therapist) to allow the user to make a selection from the library for playback. In other embodiments, in addition to or as an alternative to a built-in audio playback system, the system 300 may include an auxiliary audio input to allow the system 300 to receive input from a secondary playback system, such as a personal music playback device (e.g. an iPod, MP3 player, smart phone, or the like). In some embodiments, in addition to or as an alternative to the above auditory input, the system 300 may include a microphone or like means to allow the system 300 to receive auditory input from ambient sound, such as a live musical performance or music broadcast from secondary speakers, such as the user's home stereo system. In embodiments where the audio signal 304 is received by the system through a built-in playback system or auxiliary input such as through a MP3 player, the system may further comprise headphones or integrated speakers to allow the listener to hear the audio signal 304 in real time.

The system 300 may include a profile manager 306. The profile manager 306 may be or include a processor or internet-enabled software application accessing non-transitory and/or random-access memory which stores data pertaining to one or more users or patients, such as identifying information (e.g. name or patient ID number) stored information from previous therapies, and/or a library of audio files, in addition to various user preferences, such as song selection. The profile manager 306 may be communicably coupled with the AAS 302, to facilitate selection, management, or otherwise control of the auditory input or audio signals.

In some embodiments, the profile manager 306 may provide a user interface for prompting a user to choose his or her own individualized music preferences as an auditory stimulus. Such implementations can maximize effectiveness of the given system by stimulating auditory and reward systems in patients with early stages of dementia and cognitive decline.

The system 300 may include an Entrainment Simulator(ES) 308. The ES 308 may receive and process the received audio signal(s) (e.g., from the AAS 302), to simulate processing in the human brain. The ES 308 may simulate processing of the audio signals, to suggest and output oscillation signals to enhance the received audio signal(s) and thereby enhance the therapeutic effect of the treatment. In some embodiments, the AAS 302 is operatively connected to the ES 308 and provides data to the ES 308 in the form of an onset signal. In some embodiments, the ES 308 also interfaces with the profile manager 306 to, e.g., recall patient data from prior therapies. In some embodiments, the ES 308 may simulate entrained neural oscillations to predict the frequency, phase, and amplitude of the human neural response to music.

The ES 308 may include one or more oscillatory neural networks designed to simulate neural entrainment. In embodiments, an artificial oscillatory neural network receives a preprocessed an auditory stimulus (music), and entrains simulated neural oscillations to predict the frequency, phase, and relative amplitudes of the human neural response to the music. In some embodiments, the ES 308 may include a deep neural network, an oscillator network, a set of numerical formulae, an algorithm, or any other component configured to mimicking an oscillatory neural network. The ES 308 can be configured predict the frequencies, phases, and relative amplitudes of oscillations in the typical human brain that are entrained and driven by any given musical stimulus. The ES 308 can be configured to predict responses in at least the delta (1-4 Hz), theta (4-8 Hz) and low gamma (30-50 Hz) frequency bands.

The system 300 may include an Oscillation Selection Module (OSM) 310. The OSM 310 may be communicably coupled to the ES 308. The OSM 310 may receive the input from the ES 308, and outputs one or more selected oscillation states as frequencies, amplitude, and phases, for visual stimulation. The OSM 310 may be configured to select the most prominent oscillations in one or more predetermined frequency ranges (in preferred embodiments, the delta, theta, and gamma frequency bands) for visual stimulation. In some embodiments, the OSM 310 may couples the visual gamma frequency stimulation to the beat and rhythmic structure of music through phase-amplitude coupling. The OSM 310 may select variable, music-based frequencies in the delta, theta and gamma ranges for visual stimulation to the user, which stimulation is produced by a Brain Rhythm Stimulator, as described below

The system 300 may include a brain rhythm stimulator (BRS) 312. The BRS 312 may be configured to generate, produce, or otherwise provide a control signal for an output device 314, to provide audio and/or visual stimulation, based on data from the OSM 310, ES 308, and/or AAS 302. The BRS 312 may be configured to use the simulated neural oscillations to synchronize visual stimulation in the selected frequency ranges to the rhythm of music via the output device 314, such as an LED light ring, as described below. In some embodiments, the BRS 312 may output rhythmic visual stimulation to the user. The BRS 312 can include a pattern buffer, a generation module, adjustment module, and a filtering component, and may be operatively connected to an output device 314 comprising a means of displaying rhythmic light stimulation. The BRS 314 can also interface with the profile manager 306 which stores data pertaining to one or more users or patients. Thus, in some embodiments, information stored by the profile manager 306 may also include previously-captured or user-selected preferences of patterns, waveforms or other parameters of stimulation, such as colors, preferred by the user/patient.

The output device 314 may include LED lights, a computer monitor, a TV monitor, goggles, virtual reality headsets, augmented reality glasses, smart glasses, or other suitable stimulation output devices. In some embodiments the output device 314 may be a stimulation unit for generating tactile, vibratory, thermal and/or electrical transcutaneous stimuli, such as in a wearable device, smart watch, or mobile device. In some embodiments, the output device 314 may include a stimulation unit for generating electromagnetic fields or electrical currents, such as an array of electromagnets or electrodes, to deliver transcranial stimulation.

Collectively, the BRS may be configured to (1) read the patient's profile from the profile manager, (2) select a pattern based on the profile, (3) retrieve one or more selected oscillatory signals and/or states from the ES/OSM, (4) generate a pattern, (5) adjust the pattern based on the profile, and (5) display or output the rhythmic stimulation on an output device. In some embodiments, a pattern refers to a light pattern, and an output device refers to a visual output device.

The system 300 may include a Brain Oscillation Monitor (BOM) 316. The BOM 316 may provide neural feedback that can be used to optimize the frequency, amplitude, and phase of the visually presented oscillations, so as to optimize the frequency, phase, and amplitude of the oscillations in the brain. In some embodiments, the BOM 316 may provide feedback to the system 300 (e.g., to the ES 308), such that the ES 308 can adjust parameters to optimize the phase of outgoing oscillation signals. The BOM 316 can include, interface with, or otherwise communicate with electrodes, magnetometers, or other components arranged to sense brain activity, a signal amplifier, a filtering component, and a feedback interface component. In some embodiments, the BOM 316 can provide feedback in the form of EEG signals to the ES 308. The BOM 316 may be configured to identify the frequency, phase, and amplitude of brain oscillations entrained by the stimulus. The BOM 316 may be configured to sense electrical or magnetic fields in the brain, amplify the brain signal, filter the signal to identify specific neural frequencies, and provide input to the ES 308 as set forth above. The BOM 316 may be configured to sense electrical or magnetic fields in the brain can include electrodes connected to an electroencephalogram (EEG), intracranial EEG (iEEG), also known as electrocorticography (ECoG), magnetoencephalography (MEG), and other system for sensing electrical or magnetic fields.

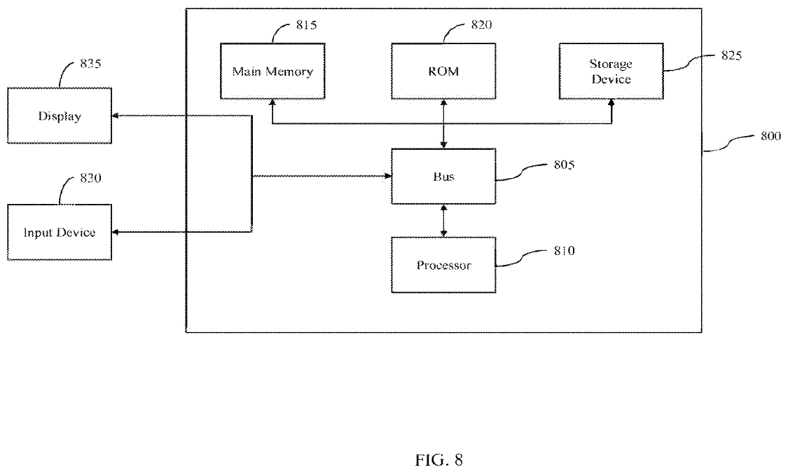

The AAS 302, profile manager 306, ES 308, OSM 310, BRS 312, and BOM 316 may each be or include any hardware, including processors, circuitry, or any other processing components, including any of the hardware or components described below with reference to FIG. 8.

Collectively, the system 300 may be configured to (1) receive auditory input, (2) simulate neural entrainment to the pre-processed auditory signal using one or more Entrainment Simulator(s) 308, which may include multi-frequency artificial neural oscillator networks, (3) couple oscillations within the networks using phase-amplitude or phase-phase coupling, (4) use adaptive learning algorithms to adjust coupling parameters and/or intrinsic parameters, and/or (5) select the most prominent oscillations in one or more frequency bands for display as a visual stimulus, via the BRS 312, described below.

In various embodiments, the rhythmic visual stimulus selected for output to the user (as described below) may include delta, theta, and/or gamma frequencies, as well as theta-gamma and/or delta-gamma phase-amplitude coupling, to enhance naturally occurring oscillatory responses to musical rhythm. The sensory cortices (e.g. primary visual and primary auditory cortices) in the brain are functionally connected to areas important for learning and memory, such as the hippocampus and the medial and lateral prefrontal cortices. Thus, coupling a complex rhythmic visual stimulus, including delta, theta, and gamma-frequency visual stimulation to musical rhythm can drive theta, gamma, and theta gamma coupling in the brain, activating neural circuitry involved in learning, memory, and cognition. This, in turn, can drive learning and memory circuits involved in music.

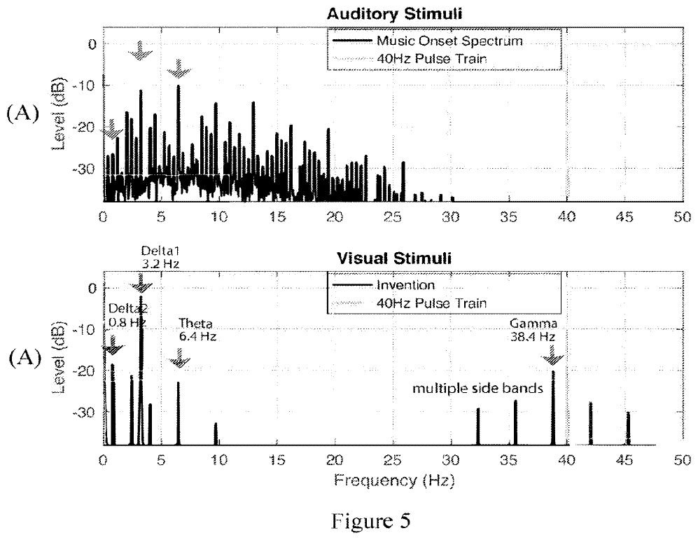

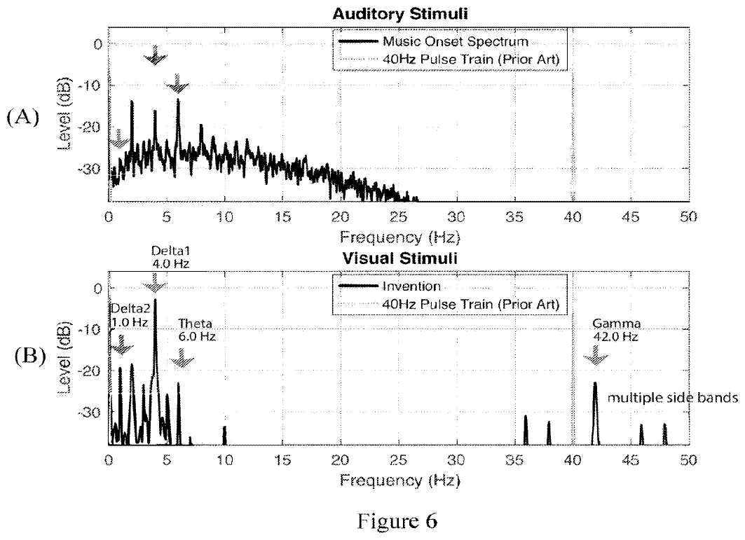

Referring now to FIG. 5 and FIG. 6, depicted are diagrams showing example stimuli using different songs and visual stimulus, according to example implementations of the present disclosure. Specifically, FIG. 5 and FIG. 6 show comparisons between the auditory and visual stimulus provided by the systems and methods described herein as compared with a 40 Hz pulse train. FIG. 5 and FIG. 6 illustrate the diverse frequencies of audio and visual stimuli provided by both the systems and methods of the present disclosure and a 40 Hz pulse train. FIG. 5 and FIG. 6 each illustrate a stimulus provided by a different song. As can be seen, a 40 Hz pulse train provides both audio and visual stimulation at a single frequency, which can easily be contrasted with the broad range of frequencies at which the systems and methods described herein both audio and visual stimulation.

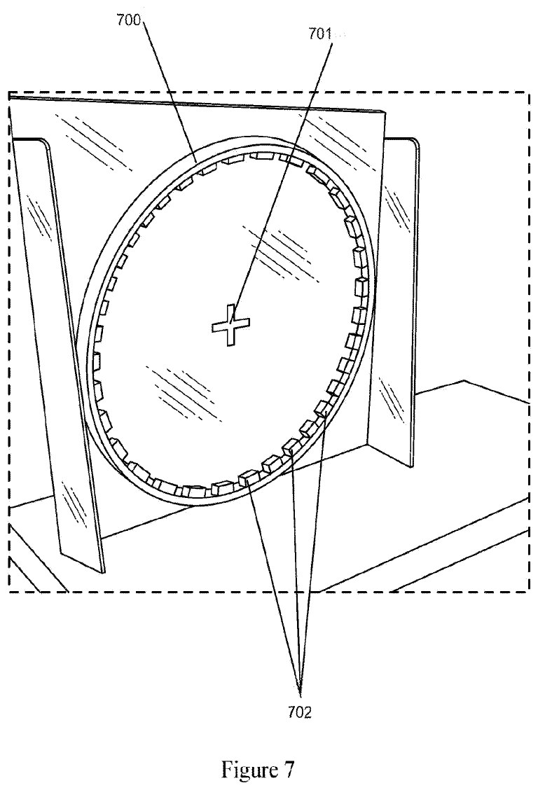

Referring now to FIG. 7, depicted is one example of an output device 314 for providing visual stimulation. The output device 314 is provided via a visual stimulation ring 700 comprising LED lights 702 that are operatively connected to the system 300 including the BRS 312. In some embodiments, the visual stimulation ring 700 is positioned in front of the participant, who is asked to focus on the center, indicated by reference character 701. In some embodiments, the visual stimulation ring 700 is placed at the appropriate distance to stimulate the retina at a specific visual angle. For example, the ring 700 may be placed at the appropriate distance to stimulate the retina at a visual angle of between 0 and 15 degrees, or between 10 and 60 degrees, or between 15 and 50 degrees, or between 15 and 25 degrees, or between 18 and 22 degrees, or between 19 and 21 degrees. In some embodiments, the visual stimulation ring 700 may be placed at the appropriate distance to stimulate the retina at a visual angle of 20 degrees where the maximum density of rods is found in the retina.

While illustrated as a stimulation ring 700, various other output devices 314 may be used as part of the system 300, either together with the stimulation ring 700 or to supplement the stimulation ring 700. For example, and in some embodiments, the output device 314 may include a head wearable device. The head wearable device may include a display and/or one or more speakers of a speaker system. The head wearable device may include augmented reality glasses, virtual reality goggles, etc. The display of the head wearable device may render the visual pattern to the user. For instance, where the head wearable device includes augmented reality glasses, the augmented reality glasses may augment the environment of the user visible through the glasses with the visual pattern. As another example, where the head wearable device includes virtual reality goggles [or other non-AR goggles), the goggles may display the visual pattern on displays adjacent to the patient's eyes. In some embodiments, the display of the head wearable device may display separate visual patterns on each eye of the patient, and at different angles, to provide visual stimulation to the patient. The one or more speakers may include in-car speakers or ear buds for each car of the patient, headphones, a speaker system (e.g., locally on the head wearable device), etc. The one or more speakers may be configured to render the audio signal 304, to provide audio stimulation to the patient.

In some embodiments, the output device 314 may include a plurality of output devices 314. For example, the output device 314 may include an audio output device 314 and a visual output device 314. The audio output device 314 may be configured to receive a control signal from the BRS 312 for rendering the audio signal 304 to the patient as audio stimulation. Similarly, the visual output device 314 may be configured to receive a control signal from the BRS 312 for rendering a visual pattern to the patient as visual stimulation. The audio output device 314 may be or include headphones, ear buds, a speaker system, etc. The visual output device 314 may include the stimulation ring 700, a display device (e.g., a television, a tablet, smartphone, or other display), a head wearable device including a display, and so forth.

Accordingly, in a method according to one embodiment of the present solution, the system may perform the processes of:
(A) receiving an auditory input,
(B) filtering the acoustic signal,
(C) detecting the onset of acoustic events,
(D) simulating neural entrainment to the pre-processed auditory signal using one or more multi-frequency neural oscillator networks,
(E) coupling oscillations within the networks using phase-amplitude or phase-phase coupling,
(F) using adaptive learning algorithms to adjust coupling parameters and/or intrinsic parameters,
(G) selecting the most prominent oscillations in the delta, theta, and/or gamma frequency bands for display,
(H) generating a light pattern, and
(I) displaying the rhythmic light on a visual output device.

In some embodiments, prior to receiving an audio input, the system may perform the processes of prompting the user to select a source of audio input and/or to make a selection from a library of songs or musical compositions stored by the system.

Self-selected music, that is, music that an individual patient has selected and which he/she is familiar with, may be more effective at engaging larger networks of brain activity compared to music selected by others, or music that the patient is not familiar with, in regions of the brain that include the hippocampus as well as the auditory cortex and the frontal lobe regions that are important for long-term memory. As such, listening to familiar music may be more effective at driving brain activity in older adults, and it activates more brain areas. Importantly, familiar music may drive greater activation in the hippocampus, a key region for memory.

Music selected by the listener may be more likely to be well-liked and familiar to the listener and may be more effective at engaging brain activity than music that is selected by researchers. In particular, self-selected music may increase activity in the dopaminergic reward system, in the default mode network, and in predictive processes of the brain, in addition to activating the auditory system. Prolonged music listening may also increase the functional connectivity of the brain from sensory cortices towards the dopaminergic reward system, which is responsible for a variety of motivated behaviors.

Therefore, in some embodiments, the auditory stimulus may include music, which is self-selected by patients, which has the practical impact of maximizing engagement throughout the brain. The systems and methods described herein may facilitate reception of musical recordings from patients while the patients are simultaneously watching captivating, audiovisual displays that include delta, theta gamma-frequency stimulation, further improving patient compliance with the disclosed treatment protocol(s).

In some embodiments, prior to the processes of generating and displaying a light pattern, the system 300 may prompt the user to select a profile from an input device and/or user interface integrated in or coupled with the system 300. The system 300 may perform one or more of the following processes: (G2) read the patient's profile from the profile manager 306, (G3) select a light pattern based on the profile, (G4) retrieve one or more oscillatory signals from the ES 308, (H) generate a light pattern, and (H2) adjusts the light pattern based on the profile.

In some embodiments, the system 300 may also optimize the frequency, phase, and/or amplitude of outgoing oscillation signals based on data received from the BOM 316. Accordingly, the system 300, on an intermittent or ongoing basis, may perform one or more of the following additional processes: (J) receive input from the BOM 316, (K) provide input to the ES 308, (L) couple input through phase-phase coupling, and (M) use adaptive learning algorithms to adjust coupling parameters and/or intrinsic parameters to optimize the frequency, phase, and amplitude of outgoing oscillation signals.

Thus, the systems and methods of the present solution may provide neural stimulation to a user via at least a presentation of rhythmic visual stimulation simultaneously, synchronously and in coordination with, musical stimulation.

For example, in some embodiments, the system 300 may generate and display light patterns based on system self-selection or on profile data housed for an individual user to be displayed simultaneously with musical stimulation. In some embodiments, the system 300 may perform one or more of the following additional processes:
(A) select one or more oscillations in the delta, theta, and/or gamma frequency bands,
(B) generate a light pattern using the one or more oscillations selected, and
(C) display said light pattern on the visual output device 314.

The system 300 may also consult a user's profile and selects a light pattern based on the profile. The system 300 may first prompt the user to select a profile from an input device and/or user interface integrated in or coupled with the system 300, and read the patient's profile from the profile manager 306 in order to determine the proper light pattern to display.

As described herein, and in some embodiments, the AAS 302 may receive auditory input through a microphone or auxiliary audio input, filter the acoustic signal, detect onset of acoustic events (e.g., notes or drum hits), and adjust the gain of the resulting signal.

As described herein, and in some embodiments, the ES 308 may receive auditory input from the AAS 302, simulates neural entrainment to the pre-processed auditory signal using one or more multi-frequency neural oscillator networks using said input, couple oscillations within the networks using phase-amplitude or phase-phase coupling, use adaptive learning algorithms to adjust coupling parameters and/or intrinsic parameters, and select oscillations for display in the predetermined frequency ranges, based on a retrieved profile. The ES 308 may also receive input from the BOM 316, provide input to one or more multi-frequency neural networks, couple neural input through phase-phase coupling, and use adaptive learning algorithms to adjust coupling parameters to optimize the amplitude and phase of outgoing oscillation signals.

As described herein, and in some embodiments, the BRS 312 may read the patient's profile from the profile manager 306, select a light pattern based on the profile, read one or more oscillatory signals from the ES 308, select at least one of a delta frequency, a theta frequency, a gamma frequency, and or a combination of frequencies, whose frequencies, amplitudes and phases are determined by the ES 308, generate a rhythmic light pattern based on the selected frequencies, adjust the light pattern based on the profile, and display rhythmic visual stimulation on LEDs, a computer monitor, a TV monitor, or other suitable light output device, which is directed toward the eye.

The result of the systems and methods described herein may be that the system senses electrical or magnetic fields in the brain, amplifies the brain signal, and filters the signal to identify specific neural frequencies. In some embodiments, the system then collects output from the user's brain based on the brain's receipt of the visual and audio stimulation, and returns this feedback to the ES 308 to further optimize the visual and audio stimulation.

The system and methods can entrain and drive oscillatory neural activity that is involved in learning, memory, and cognition. By providing music as the sole auditory stimulus, plus visual stimulation in the delta, theta, and/or gamma frequency bands, the system and methods can serve as a method for treating, preventing, protecting against or otherwise affecting Alzheimer's Disease and dementia.

In various instances, where a patient is undergoing treatment or is otherwise undergoing both audio and visual stimulation as described herein, often that stimulation is at a targeted or particular frequency or frequency band (e.g., in the delta, theta, and/or gamma band) to stimulate a particular portion of the patient's brain. For example, and as described above, However, even if the audio stimulation is at the same frequency as the visual stimulation, the resultant brain signals from the audio stimulation and visual stimulation may not arrive at the target portion of the patient's brain at the same time. In this regard, while the audio and visual stimulation may be at substantially the same frequency, the audio and visual stimulations may be out of phase. To the extent that the audio and visual stimulations are out of phase, the resultant brain signals may destructively interfere with each other, thereby reducing the efficacy of stimulation and treatment. On the other hand, to the extent that the audio and visual stimulations are in phase with one another, the resultant brain signals may constructively interfere with each other, thereby amplifying the stimulation in the target portion of the patient's brain.

According to the systems and methods described herein, and as described in greater detail below, the ES 308 may be configured to modify, update, change, or otherwise shift a phase of the audio and/or visual stimulation, to phase align the audio stimulation and the visual stimulation. By shifting a phase of the audio and/or visual stimulation such that the resultant brain signals constructively interfere with one another, the systems and methods described herein may amplify the stimulation in the target portion of the patient's brain, thereby increasing the efficacy of stimulation and treatment.

In some embodiments, the ES 308 may be configured to shift the phase of the audio and/or visual stimulation, based on feedback identified or otherwise provided by the BOM 316. As described above, the BOM 316 can include, interface with, or otherwise communicate with electrodes, magnetometers, or other components arranged to sense brain activity, a signal amplifier, a filtering component, and a feedback interface component. The BOM 316 can thus sense brain activity (such as the resultant brain signals from both audio and visual stimulation) of the patient undergoing neural stimulation. The BOM 316 can sense the brain activity in real-time (or near-real-time) as the audio stimulation, including the target frequencies amplified or added thereto, and visual stimulation, including those same target frequencies, are provided to the patient. In this regard, the BOM 316 can measure, quantify, or otherwise sense the brain activity (e.g., as EEG signals, iEEG signals, ECoG signals, MEG signals, or other electrical or magnetic field signals) in response to the audio and visual stimulation. The BOM 316 can transmit, send, or otherwise provide the sensed brain activity as feedback to the ES 308. The BOM 315 can provide, for example, EEG signals as feedback to the ES 308.

The ES 308 may be configured to maintain, include, or otherwise access one or more models corresponding to a target brain activity in response to audio and/or visual stimulation. In some embodiments, the ES 308 may be configured to maintain a plurality of models, where each model corresponds to a particular target brain activity for a particular targeted frequency or frequency band. For example, the ES 308 may be configured to maintain models which include a representative EEG signal for audio and visual stimulation in the delta frequency band, a representative EEG signal for audio and visual stimulation in the theta frequency band, a representative EEG signal for audio and visual stimulation in the gamma frequency band, and various combinations thereof (e.g., delta-theta, delta-gamma, theta-gamma, delta-theta-gamma, etc.). In some embodiments, the ES 308 may maintain the models locally and/or at one or more remote storage locations (e.g., similar to the profiles stored or otherwise maintained by the profile manager 306). The ES 308 may be configured to maintain the models with a tag, indicator, or identifier which identifies a type of stimulation corresponding to the model.

The ES 308 may be configured to identify a model of the one or more models which corresponds to the audio and visual stimulation provided to the user (e.g., via the output device 314) and selected by the BRS 312. For example, where the audio and visual stimulation is in the delta frequency band, the ES 308 may be configured to identify the model which corresponds to the delta frequency band type of stimulation (e.g., by identifying the tag or identifier for the model which identifies the type of stimulation as the delta frequency band). Additionally, where the audio and visual stimulation is in the theta or gamma frequency band, the ES 308 may be configured to identify the model which corresponds to those particular types of stimulation.

The ES 308 may be configured to compare the feedback signal received from the BOM 316 to the model identified and corresponding to the type of audio and visual stimulation. The ES 308 may be configured to compare an amplitude of the feedback signal to an amplitude of the target brain activity for the particular type of audio and visual stimulation. The ES 308 may be configured to determine a difference between the amplitudes. The ES 308 may be configured to determine, detect, or otherwise identify a destructive interference condition based on the difference between the amplitudes. For example, the ES 308 may be configured to identify the destructive interference condition responsive to the difference satisfying a threshold criteria (e.g., being greater than or equal to a threshold difference). Where the ES 308 determines that the difference is less than the threshold difference, that result may be due to various conditions including, for example, reduced brain responses due to various neurological conditions. However, where the ES 308 determines that the difference satisfies the threshold criteria, that result may be due to the resultant brain signal from the audio stimulation arriving at the target brain region at a different time than the resultant brain signal from the visual stimulation. In other words, where the ES 308 determines that the difference satisfies the threshold criteria, that result may be due to resultant brain signals being phase shifted from one another, thereby causing destructive interference.

Where the difference between the amplitude of the feedback signal and the amplitude of the model corresponding to the type of audio and visual stimulation satisfies the threshold criteria, the ES 308 may be configured to apply a phase shift to one of the audio stimulation or visual stimulation. The ES 308 may be configured to apply a phase shift to the audio or visual stimulation by delaying an onset of the audio stimulation or visual stimulation. For example, assuming that the visual stimulation is a light pattern at a particular frequency, that ES 308 may be configured to apply a phase shift to the visual stimulation by delaying onset of the light pattern. The ES 308 may be configured to apply the phase shift while maintaining the frequency of the audio or visual stimulation. In some embodiments, the ES 308 may be configured to apply the phase shift to both the audio stimulation and the visual stimulation. For example, the ES 308 may be configured to apply the phase shift to delay onset of the audio stimulation a first duration, and apply another phase shift to delay onset of the visual stimulation a second duration. In some embodiments, the ES 308 may be configured to apply the phase shift to one of the audio stimulation or the visual stimulation. For example, the ES 308 may be configured to maintain the audio stimulation at the same phase and frequency, but may apply a phase shift to the visual stimulation (or vice versa).

The ES 308 may be configured to receive subsequent feedback from the BOM 316 responsive to applying the phase shift to the audio and/or visual stimulation (and the output device 314 outputting the audio or visual stimulation to the user). The ES 308 may be configured to receive the subsequent feedback from the BOM 316 as the output device 314 outputs the audio and/or visual stimulation following the ES 308 applying the phase shift to one or more of the audio or visual stimulation. Similar to the initial feedback, the ES 308 may be configured to compare an amplitude of the subsequent feedback to the model for the particular type of audio or visual stimulation. The ES 308 may apply additional phase shifts to the audio and/or visual stimulation until the difference between the amplitudes are greater than (or equal to) a threshold, thereby indicating the audio and visual stimulation are constructively interfering with one another.

In some embodiments, the ES 308 may be configured to determine, detect, or otherwise identify amplitudes at the target frequencies using the feedback from the BOM 316. In some embodiments, the ES 308 may be configured to identify the amplitudes from the feedback following the phase shift being applied to the visual stimulation (and/or audio stimulation). The ES 308 may be configured to compare the amplitude at the target frequencies to a threshold amplitude. To the extent that the amplitude at the target frequencies in the feedback from the BOM 316 does not satisfy the threshold amplitude (e.g., is less than or equal to the threshold amplitude), the ES 308 may be configured to increase an amplitude of the visual stimulation (e.g., at the output device 314 side). For example, the ES 308 may be configured to increase the amplitude of the visual stimulation at the output device 314 side by increasing an intensity of the light pattern, a brightness, etc. The ES 308 may be configured to increase the amplitude at the input side, to cause an increased amplitude at the target frequency in the feedback signal from the BOM 316. While described as increasing the amplitude after applying a phase shift, in various embodiments, the ES 308 may be configured to perform amplitude increase and phase shifting in parallel (e.g., at substantially the same time or at the same time), the ES 308 may be configured to perform the amplitude increase prior to applying the phase shift, etc. Such implementations and embodiments may provide for increased stimulation at the target frequencies in the target portion of the brain, thereby increasing the efficacy of treatment from such stimulation.

FIG. 8 depicts an example block diagram of an example computer system 800. The computer system or computing device 800 can include or be used to implement a data processing system or its components. The computing system 800 includes at least one bus 805 or other communication component for communicating information and at least one processor 810 or processing circuit coupled to the bus 805 for processing information. The computing system 800 can also include one or more processors 810 or processing circuits coupled to the bus for processing information. The computing system 800 also includes at least one main memory 815, such as a random access memory (RAM) or other dynamic storage device, coupled to the bus 805 for storing information, and instructions to be executed by the processor 810. The main memory 815 can be used for storing information during execution of instructions by the processor 810. The computing system 800 may further include at least one read only memory (ROM) 820 or other static storage device coupled to the bus 805 for storing static information and instructions for the processor 810. A storage device 825, such as a solid state device, magnetic disk or optical disk, can be coupled to the bus 805 to persistently store information and instructions.

The computing system 800 may be coupled via the bus 805 to a display 835, such as a liquid crystal display, or active matrix display, for displaying information to a user. An input device 830, such as a keyboard or voice interface may be coupled to the bus 805 for communicating information and commands to the processor 810. The input device 830 can include a touch screen display 835. The input device 830 can also include a cursor control, such as a mouse, a trackball, or cursor direction keys, for communicating direction information and command selections to the processor 810 and for controlling cursor movement on the display 835.

The processes, systems and methods described herein can be implemented by the computing system 800 in response to the processor 810 executing an arrangement of instructions contained in main memory 815. Such instructions can be read into main memory 815 from another computer-readable medium, such as the storage device 825. Execution of the arrangement of instructions contained in main memory 815 causes the computing system 800 to perform the illustrative processes described herein. One or more processors in a multi-processing arrangement may also be employed to execute the instructions contained in main memory 815. Hard-wired circuitry can be used in place of or in combination with software instructions together with the systems and methods described herein. Systems and methods described herein are not limited to any specific combination of hardware circuitry and software.

Although an example computing system has been described in FIG. 8, the subject matter including the operations described in this specification can be implemented in other types of digital electronic circuitry, or in computer software, firmware, or hardware, including the structures disclosed in this specification and their structural equivalents, or in combinations of one or more of them.

Having now described some illustrative implementations, it is apparent that the foregoing is illustrative and not limiting, having been presented by way of example. In particular, although many of the examples presented herein involve specific combinations of method acts or system elements, those acts and those elements can be combined in other ways to accomplish the same objectives. Acts, elements and features discussed in connection with one implementation are not intended to be excluded from a similar role in other implementations or implementations.

The hardware and data processing components used to implement the various processes, operations, illustrative logics, logical blocks, modules and circuits described in connection with the embodiments disclosed herein may be implemented or performed with a general purpose single-or multi-chip processor, a digital signal processor (DSP), an application specific integrated circuit (ASIC), a field programmable gate array (FPGA), or other programmable logic device, discrete gate or transistor logic, discrete hardware components, or any combination thereof designed to perform the functions described herein. A general purpose processor may be a microprocessor, or, any conventional processor, controller, microcontroller, or state machine. A processor also may be implemented as a combination of computing devices, such as a combination of a DSP and a microprocessor, a plurality of microprocessors, one or more microprocessors in conjunction with a DSP core, or any other such configuration. In some embodiments, particular processes and methods may be performed by circuitry that is specific to a given function. The memory (e.g., memory, memory unit, storage device, etc.) may include one or more devices (e.g., RAM, ROM, Flash memory, hard disk storage, etc.) for storing data and/or computer code for completing or facilitating the various processes, layers and modules described in the present disclosure. The memory may be or include volatile memory or non-volatile memory, and may include database components, object code components, script components, or any other type of information structure for supporting the various activities and information structures described in the present disclosure. According to an exemplary embodiment, the memory is communicably connected to the processor via a processing circuit and includes computer code for executing (e.g., by the processing circuit and/or the processor) the one or more processes described herein.

The present disclosure contemplates methods, systems and program products on any machine-readable media for accomplishing various operations. The embodiments of the present disclosure may be implemented using existing computer processors, or by a special purpose computer processor for an appropriate system, incorporated for this or another purpose, or by a hardwired system. Embodiments within the scope of the present disclosure include program products comprising machine-readable media for carrying or having machine-executable instructions or data structures stored thereon. Such machine-readable media can be any available media that can be accessed by a general purpose or special purpose computer or other machine with a processor. By way of example, such machine-readable media can comprise RAM, ROM, EPROM, EEPROM, or other optical disk storage, magnetic disk storage or other magnetic storage devices, or any other medium which can be used to carry or store desired program code in the form of machine-executable instructions or data structures and which can be accessed by a general purpose or special purpose computer or other machine with a processor. Combinations of the above are also included within the scope of machine-readable media. Machine-executable instructions include, for example, instructions and data which cause a general purpose computer, special purpose computer, or special purpose processing machines to perform a certain function or group of functions.

The phraseology and terminology used herein is for the purpose of description and should not be regarded as limiting. The use of “including” “comprising” “having” “containing” “involving” “characterized by” “characterized in that” and variations thereof herein, is meant to encompass the items listed thereafter, equivalents thereof, and additional items, as well as alternate implementations consisting of the items listed thereafter exclusively. In one implementation, the systems and methods described herein consist of one, each combination of more than one, or all of the described elements, acts, or components.

Any references to implementations or elements or acts of the systems and methods herein referred to in the singular can also embrace implementations including a plurality of these elements, and any references in plural to any implementation or element or act herein can also embrace implementations including only a single element. References in the singular or plural form are not intended to limit the presently disclosed systems or methods, their components, acts, or elements to single or plural configurations. References to any act or element being based on any information, act or element can include implementations where the act or element is based at least in part on any information, act, or element.

Any implementation disclosed herein can be combined with any other implementation or embodiment, and references to “an implementation,” “some implementations,” “one implementation” or the like are not necessarily mutually exclusive and are intended to indicate that a particular feature, structure, or characteristic described in connection with the implementation can be included in at least one implementation or embodiment. Such terms as used herein are not necessarily all referring to the same implementation. Any implementation can be combined with any other implementation, inclusively or exclusively, in any manner consistent with the aspects and implementations disclosed herein.

Where technical features in the drawings, detailed description or any claim are followed by reference signs, the reference signs have been included to increase the intelligibility of the drawings, detailed description, and claims. Accordingly, neither the reference signs nor their absence have any limiting effect on the scope of any claim elements.

Systems and methods described herein may be embodied in other specific forms without departing from the characteristics thereof. References to any terms of degree include variations of +/−10% from the given measurement, unit, or range unless explicitly indicated otherwise. Coupled elements can be electrically, mechanically, or physically coupled with one another directly or with intervening elements. Scope of the systems and methods described herein is thus indicated by the appended claims, rather than the foregoing description, and changes that come within the meaning and range of equivalency of the claims are embraced therein.

The term “coupled” and variations thereof includes the joining of two members directly or indirectly to one another. Such joining may be stationary (e.g., permanent or fixed) or moveable (e.g., removable or releasable). Such joining may be achieved with the two members coupled directly with or to each other, with the two members coupled with each other using a separate intervening member and any additional intermediate members coupled with one another, or with the two members coupled with each other using an intervening member that is integrally formed as a single unitary body with one of the two members. If “coupled” or variations thereof are modified by an additional term (e.g., directly coupled), the generic definition of “coupled” provided above is modified by the plain language meaning of the additional term (e.g., “directly coupled” means the joining of two members without any separate intervening member), resulting in a narrower definition than the generic definition of “coupled” provided above. Such coupling may be mechanical, electrical, or fluidic.

References to “or” can be construed as inclusive so that any terms described using “or” can indicate any of a single, more than one, and all of the described terms. A reference to “at least one of ‘A’ and ‘B”’ can include only ‘A’, only ‘B’, as well as both ‘A’ and ‘B’. Such references used in conjunction with “comprising” or other open terminology can include additional items.

Modifications of described elements and acts such as variations in sizes, dimensions, structures, shapes and proportions of the various elements, values of parameters, mounting arrangements, use of materials, colors, orientations can occur without materially departing from the teachings and advantages of the subject matter disclosed herein. For example, elements shown as integrally formed can be constructed of multiple parts or elements, the position of elements can be reversed or otherwise varied, and the nature or number of discrete elements or positions can be altered or varied. Other substitutions, modifications, changes and omissions can also be made in the design, operating conditions and arrangement of the disclosed elements and operations without departing from the scope of the present disclosure.

What is claimed is:

1. A system comprising:
an output device configured to output an audio signal for audio stimulation of the patient, and a visual pattern for video stimulation of the patient;
a brain oscillation monitor configured to generate feedback indicative of a response to the audio stimulation and the visual stimulation; and
one or more processors configured to:
determine a target frequency for the audio stimulation and the visual stimulation; and
cause constructive interference of the audio stimulation and the visual stimulation output by the output device at the target frequency, by modifying at least one of a phase of the visual pattern or an amplitude of onsets for the visual pattern, relative to the audio signal, according to the feedback from the brain oscillation monitor.

2. The system of claim 1, wherein the one or more processors are configured to determine, using the feedback from the brain oscillation monitor, an amplitude in the response at the target frequency.

3. The system of claim 2, wherein the one or more processors are configured to modify the at least one of the phase or the amplitude, responsive to the amplitude in the response at the target frequency satisfying a threshold criteria.

4. The system of claim 1, wherein the one or more processors are configured to modify each of the phase of the visual pattern and the amplitude of the onsets for the visual pattern, relative to the audio signal.
